(19) United States
(12) Patent Application Publication
(10) Pub. No.: US 2001/0011137 A1
(43) Pub. Date: Aug. 2, 2001

(54) Use of triphenylmethyl-1,2,3-triazoles

(76) Inventors: Klaus Urbahns, Wuppertal (DE); Frank Mauler, Overath (DE)

Correspondence Address: Kurt G. Briscoe, Norris McLaughlin & Marcus, P.A., 30th Floor, 220 East 42nd Street, New York, NY, 10017, US

(21) Appl. No.: 09/774,144

(22) Filed: Jan. 30, 2001

Related U.S. Application Data
(62) Division of application No. 09/340,930, filed on Jun. 28, 1999, now abandoned.
Application No. 09/340,930 is a division of application No. 09/119,117, filed on Jul. 20, 1998, now abandoned.

(30) Foreign Application Priority Data
Jul. 19, 1996 (DE) 19629145.3

(51) Int. Cl. C07D 249/16; C07D 403/00

(52) U.S. Cl. 548/255; 548/257

(57) ABSTRACT

The present invention relates to the use of triphenylmethyl-1,2,3-triazoles for the production of a medicament for the control of disorders of the CNS, new active compounds, processes for their preparation, and their use, in particular as cerebrally active agents.

The present invention relates to the use of triphenylmethyl-1,2,3-triazoles for the production of a medicament for the control of disorders of the CNS, new active compounds, processes for their preparation, and their use, in particular as cerebrally active agents.

It has already been disclosed that triphenylmethyl-1,2,3-triazoles can influence plant growth and have good antimycotic properties against human pathogenic and animal pathogenic fungi and yeasts and also fungicidal properties against phytopathogenic fungi [cf. DE 19 35 292, DE 19 40 626, DE 19 40 627, DE 19 40 628 and DE 24 07 305].

It has now been found that triphenylmethyl-1,2,3-triazoles of the general formula (I)

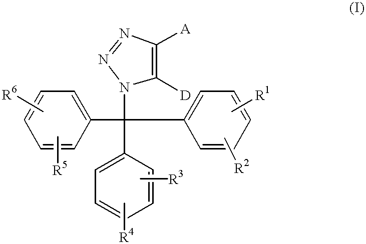

in which

A and D are identical or different and represent hydrogen, aryl having 6 to 10 carbon atoms, halogen, cyano, nitro or straight-chain or branched alkyl or alkoxycarbonyl each having up to 3 carbon atoms,

or

A and D together form a cyclic radical of the formula

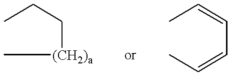

in which

a denotes the number 1 or 2,

R¹, R², R³, R⁴, R⁵ and R⁶ are identical or different and represent hydrogen, hydroxyl, halogen, nitrile, nitro, thiocyano, trifluoromethyl, straight-chain or branched alkyl, halogenoalkyl, alkoxy, alkylmercapto, alkylsulphoxy or alkylsulphonyl each having up to 6 carbon atoms, aryl or aryloxy having 6 to 10 carbon atoms or amino or dialkylamino having up to 6 carbon atoms,

and their salts,

surprisingly have a modulating action on charybdotoxin-sensitive, calcium-dependent K channels and are thus suitable for the control of disorders of the CNS.

In the context of the invention, physiologically acceptable salts are preferred. Physiologically acceptable salts are in general salts of the compounds according to the invention with inorganic or organic acids. Preferred salts are those with inorganic acids, such as hydrochloric acid, hydrobromic acid, phosphoric acid or sulphuric acid or salts with organic carboxylic or sulphonic acids, such as acetic acid, maleic acid, fumaric acid, malic acid, citric acid, tartaric acid, lactic acid, benzoic acid, or methanesulphonic acid, ethanesulphonic acid, phenylsulphonic acid, toluenesulphonic acid or naphthalenedisulphonic acid.

The compounds according to the invention can exist in stereoisomeric forms which behave either as image and mirror image (enantiomers), or which do not behave as image and mirror image (diastereomers). The invention relates both to the antipodes and the racemic forms and also the diastereomer mixtures.

Preferably, compounds of the general formula (I) in which

A and D represent hydrogen or methoxycarbonyl or together form a cyclic radical of the formula

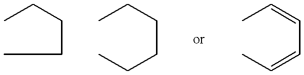

R¹, R², R³, R⁴, R⁵ and R⁶ are identical or different and represent hydrogen, nitro, fluorine, chlorine, bromine, iodine, trifluoromethyl or straight-chain or branched alkyl having up to 5 carbon atoms,

and their salts,

are used in the control of disorders of the CNS.

Particularly preferably, compounds of the general formula (I) in which

A and D represent hydrogen or methoxycarbonyl or together form a cyclic radical of the formula

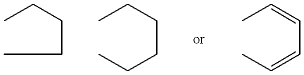

R¹, R², R³, R⁴, R⁵ and R⁶ are identical or different and represent hydrogen, nitro, fluorine, chlorine, bromine, trifluoromethyl or straight-chain or branched alkyl having up to 3 carbon atoms,

and their salts,

are used in the control of disorders of the CNS.

Very particularly preferably, compounds of the general formula (I) in which

A and D represent hydrogen,

R¹ and R² are identical or different and represent hydrogen or chlorine,

and

R³, R⁴, R⁵ and R⁶ represent hydrogen,

are used in the control of disorders of the CNS.

Likewise, very particularly preferably, the compound 1-(2-chlorotrityl)-1H-1,2,3-triazole

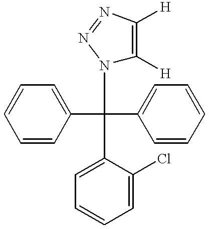

is used in the control of disorders of the CNS, in particular of stroke.

The compounds of the general formula (I) according to the invention exhibit an unforeseeable, valuable spectrum of pharmacological action.

They are modulators of charybdotoxin-sensitive calcium-dependent potassium channels (IK(Ca) channels), in particular of the central nervous system.

On account of their pharmacological properties, they can be employed for the production of medicaments for the treatment of central degenerative disorders, such as dementias, e.g. multiinfarct dementia (MID), primary degenerative dementia (PDD), presenile and senile dementia of the Alzheimer's disease type, HIV dementia and other forms of dementia, and further for the treatment of Parkinson's disease or amyotrophic lateral sclerosis and multiple sclerosis.

Furthermore, the active compounds are suitable for the treatment of brain function disorders in old age, of organic brain syndrome (OBS) and of age-related memory disorders (age-associated memory impairment, AAMI).

They are suitable for the prophylaxis, for the treatment and for the control of the sequelae of cerebral circulatory disorders, such as cerebral ischaemias, strokes, craniocerebral traumata and of subarachnoid haemorrhages.

They are useful for the treatment of depressions and psychoses, e.g. schizophrenia. They are additionally suitable for the treatment of disorders of neuroendocrine secretion and of neurotransmitter secretion and related health disorders, such as mania, alcoholism, drug abuse, addiction or disordered eating behaviour. Further areas of application are the treatment of migraine, sleep disorders and of neuropathies. They are moreover suitable as analgesics.

The active compounds are further suitable for the treatment of disorders of the immune system, in particular of T-lymphocyte proliferation and for affecting the smooth musculature, in particular of the uterus, bladder and bronchial tract, and for the treatment of related diseases, such as asthma and urinary incontinence and for the treatment of high blood pressure, arrhythmia, angina, diabetes, sickle cell anaemia, COPD (chronic obstructive pulmonary disease), cancer, restenosis and oedema formation.

The invention additionally relates to new triphenylmethyl-1,2,3-triazoles of the general formula (I), in which R³, R⁴, R⁵ and R⁶ denote hydrogen and the other substituents have the substituent meanings shown in the following table:

| R¹ | R² | A | D |
|---|---|---|---|
| 2-CH₃ | H | H | H |

| | | |
|---|---|---|
| H | H | 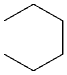 |
| 2-Cl | H | 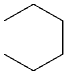 |

| | | | |
|---|---|---|---|
| H | H | CO₂CH₃ | CO₂CH₃ |

| | | |
|---|---|---|
| 3-CF₃ | H | 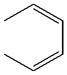 |

| | | | |
|---|---|---|---|
| 4-NO₂ | H | H | H |

The compounds of the general formula (I) can be prepared by

[A] reacting, compounds of the general formula (II)

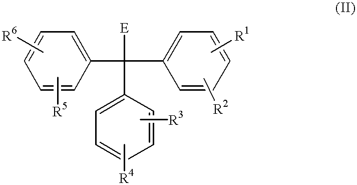

in which

R¹, R², R³, R⁴, R⁵ and R⁶ have the meanings indicated above,

and

E represents halogen, preferably chlorine,

with 1,2,3-triazole in inert solvents, if appropriate in the presence of an acid-binding agent,

or

[B] reacting compounds of the general formula (III)

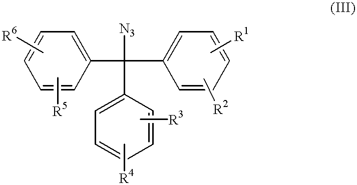

in which

R¹, R², R³, R⁴, R⁵ and R⁶ have the meanings indicated above,

with acetylene, if appropriate in the presence of a solvent.

The processes according to the invention can be illustrated by way of example by the following reaction scheme:

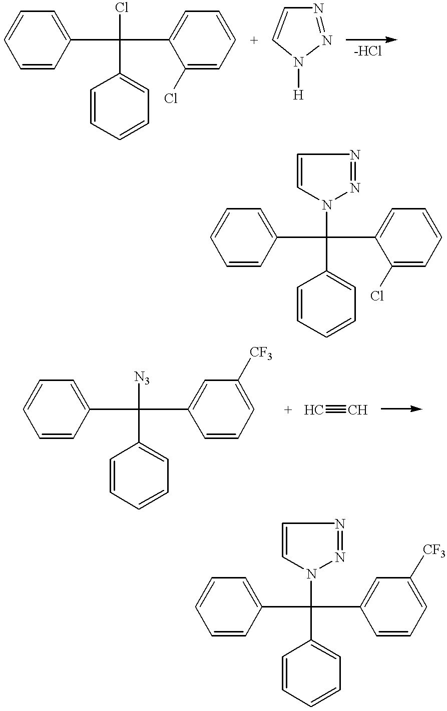

Suitable solvents in process [A] are polar organic solvents. These preferably include nitrites such as o- and p-tolunitrile and acetonitrile, ethers such as tetrahydrofuran and dioxane, sulphoxides such as dimethyl sulphoxide, amides such as dimethylformamide or hexamethylphosphoramide.

Acid binders used are inorganic or organic acid acceptors. The following may preferably be mentioned: alkali metal carbonates such as potassium carbonate and sodium carbonate, alkaline earth metal carbonates such as barium carbonate and magnesium carbonate, alkali earth metal hydroxides such as barium hydroxide and magnesium hydroxide, tertiary organic bases such as triethylamine and pyridine.

Process [A] according to the invention is in general carried out at temperatures from 60 to 150° C., preferably between 80 and 120° C., and at normal pressure.

Relative to 1 mol of trityl halide of the formula (II), preferably 1 mol of 1,2,3-triazole and 1 mol of acid acceptor are employed. However, an excess of 1,2,3-triazole (2 to 2.3 mol) can be used instead of the acid acceptor. To isolate the active compounds, the solvent is removed, and the residue is washed well with water to remove the halide formed and, if appropriate, purified by recrystallization.

Suitable solvents in process [B] likewise are polar organic solvents. These preferably include ethers such as tetrahydrofuran and dioxane, ketones such as acetone and methyl ethyl ketone, amides such as dimethylformamide and hexamethylphosphoramide and sulphoxides such as dimethyl sulphoxide.

Process [B] according to the invention is in general carried out at temperatures from 60 to 150° C., preferably between 80 and 120° C.

The reaction is in general carried out at pressures from 5 to 20 kg/cm², preferably at approximately 10 kg/cm².

Relative to 1 mol of trityl azide of the formula (III), preferably 1 mol of acetylene is employed.

The compounds of the general formulae (II) and (III) are known or can be prepared by customary methods [cf. DE 24 07 305].

The compounds of the formula (I) can also be prepared by the processes which are listed in the Offenlegungsschriften DE 19 35 292, DE 19 40 626, DE 19 40 627, DE 19 40 628 and DE 24 07 305.

### Rubidium Efflux from C6-BU1 Glioma Cells

The experiments were carried out with slight changes according to the method described by Tas et al. (Neurosci. Lett. 94, 279-284, (1988)). To this end, rat C6-BUI glioma cells are used. Detection is carried out by AAS. From the data, the increase in the efflux produced by ionomycin above the basal efflux is calculated and set as 100%. The stimulations in the presence of test substances are then related to this value.

The present invention also includes pharmaceutical preparations which, in addition to inert, non-toxic, pharmaceutically suitable auxiliaries and excipients, contain one or more compounds of the general formula (I), or which consist of one or more active compounds of the formula (I), and processes for the production of these preparations.

The active compounds of the formula (I) should be present in these preparations in a concentration from 0.1 to 99.5% by weight, preferably from 0.5 to 95% by weight of the total mixture.

In addition to the active compounds of the formula (I), the pharmaceutical preparations can also contain other pharmaceutical active compounds.

The abovementioned pharmaceutical preparations can be prepared in a customary manner according to known methods, for example with the auxiliary(ies) or excipient(s).

In general, it has proven advantageous to administer the active compound(s) of the formula (I) in total amounts of approximately 0.01 to approximately 100 mg/kg, preferably in total amounts of approximately 1 mg/kg to 50 mg/kg of body weight every 24 hours, if appropriate in the form of individual doses, to achieve the desired result.

However, if appropriate it may be advantageous to depart from the amounts mentioned, namely depending on the type and on the body weight of the subject to be treated, on individual behaviour towards the medicament, the nature and severity of the disorder, the type of preparation and administration, and the time or interval at which administration takes place.

### PREPARATION EXAMPLES

### Example 1

1-Trityl-4,5,6,7-tetrahydro-1H-benzotriazole

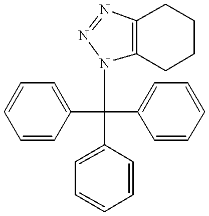

25 g (0.2 mol) of tetrahydrobenzotriazole, 55.8 g of trityl chloride and 20.2 g of triethylamine are boiled for 4 h in 250 ml of abs. acetonitrile. The solvent is stripped off and the residue is partitioned between methylene chloride and water. The organic phase is dried (Na₂SO₄), filtered and concentrated. The residue is recrystallized from acetonitrile. 45 g of the title compound are obtained.

C₂₅H₂₃N₃ (365.48)

calc.: C: 82.27 H: 6.35 found: C: 82.50 H: 6.10

The compounds shown in the following table were prepared analogously to the procedure of Example 1:

TABLE 1

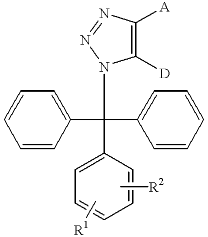

| Ex. No. | A | D | R¹/R² | M.p. (° C.) |
|---|---|---|---|---|
| 2 | H | H | 2-CH₃/H | 156 |
| 3 | H | H | 3-Br/H | 147 |
| 4 | H | H | 2-F/H | 179 |
| 5 | H | H | 3-Cl/H | 114 |
| 6 | H | H | 4-Cl/H | 160 |
| 7 | H | H | 2,4-Cl₂ | 197 |
| 8 | H | H | 3-CF₃/H | 126 |
| 9 | H | H | H/H | 187 |
| 10 | H | H | 2-Cl/H | 162 |

| | | | |
|---|---|---|---|
| 11 | 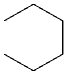 | 2-Cl/H | 196 |

| | | | | |
|---|---|---|---|---|
| 12 | COOCH₃ | COOCH₃ | H/H | 186 |

| | | | |
|---|---|---|---|
| 13 | 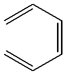 | 3-CF₃/H | 160 |

| | | | | |
|---|---|---|---|---|
| 14 | H | H | 4-NO₂/H | 132 |
| 15 | H | H | 2-iPr/H | 166 |

1. Use of compounds of the general formula (I) 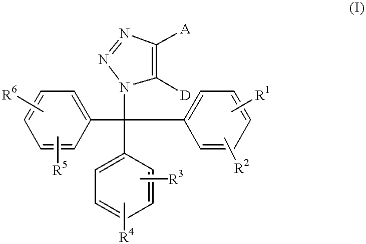
in which
A and D are identical or different and represent hydrogen, aryl having 6 to 10 carbon atoms, halogen, cyano, nitro or
straight-chain or branched alkyl or alkoxycarbonyl each having up to 3 carbon atoms,
or
A and D together form a cyclic radical of the formula 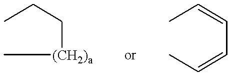
in which
a denotes the number 1 or 2,
R¹, R², R³, R⁴, R⁵ and R⁶ are identical or different and represent hydrogen, hydroxyl, halogen, nitrile, nitro, thiocyano, trifluoromethyl, straight-chain or branched alkyl, halogenoalkyl, alkoxy, alkylmercapto, alkylsulphoxy or alkylsulphonyl each having up to 6 carbon atoms, aryl or aryloxy having 6 to 10 carbon atoms or amino or dialkylamino having up to 6 carbon atoms,
and/or their salts,
for the production of a medicament for the control of disorders of the CNS.

2. Use according to claim 1, characterized in that compounds of the general formula (I) in which
A and D represent hydrogen or methoxycarbonyl or together form a cyclic radical of the formula 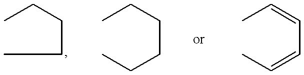
R¹, R², R³, R⁴, R⁵ and R⁶ are identical or different and represent hydrogen, nitro, fluorine, chlorine, bromine, iodine, trifluoromethyl or straight-chain or branched alkyl having up to 5 carbon atoms,
and/or their salts are employed.

3. Use according to claim 1, characterized in that compounds of the general formula (I) in which
A and D represent hydrogen or methoxycarbonyl or together form a cyclic radical of the formula 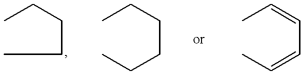
R¹, R², R³, R⁴, R⁵ and R ⁶ are identical or different and represent hydrogen, nitro, fluorine, chlorine, bromine, trifluoromethyl or straight-chain or branched alkyl having up to 3 carbon atoms,
and/or their salts are employed.

4. Use according to claim 1, characterized in that compounds of the general formula (I) in which
A and D represent hydrogen,
R¹ and R² are identical or different and represent hydrogen or chlorine,
and
R³, R⁴, R⁵ and R⁶ represent hydrogen
and/or their salts are employed.

5. Use according to claims 1 to 4, the compound of the general formula (I) being 1-(2-chlorotrityl)-1H-1,2,3-triazole 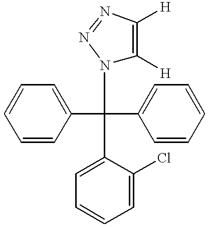
for the production of a medicament for the control of disorders of the CNS, in particular of stroke.

6. Compounds of the general formula (I) 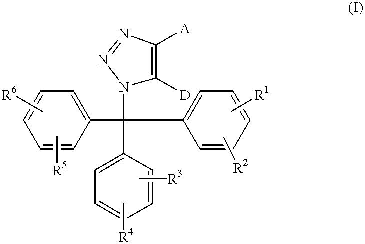
and their salts,
in which
R³, R⁴, R⁵ and R⁶ denote hydrogen,
A and D denote hydrogen,
R¹ denotes 2-CH₃ and
R² denotes hydrogen
or
R¹, R², R³, R⁴, R⁵ and R⁶ denote hydrogen and
A and D denote hydrogen or
R³, R⁴, R⁵ and R⁶ denote hydrogen,
A and D together form a cyclic radical of the formula 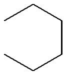
R¹ denotes 2-Cl and
R² denotes hydrogen
or
R³, R⁴, R⁵ and R⁶ denote hydrogen,
A and D denote CO₂CH₃ and
R¹ and R² denote hydrogen
or
R³, R⁴, R⁵ and R⁶ denote hydrogen,
A and D together form a radical of the formula 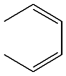
R¹ denotes 3-CF₃ and
R² denotes hydrogen
or
R³, R⁴, R⁵ and R⁶ denote hydrogen,
A and D denote hydrogen,
R¹ denotes 4-NO₂ and
R² denotes hydrogen.

7. Process for the preparation of the compounds according to claim 5, characterized in that
[A] compounds of the general formula (II) 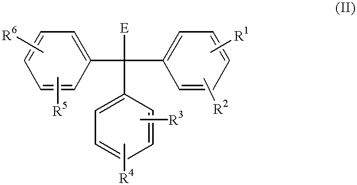
in which
R¹, R², R³, R⁴, R⁵ and R⁶ have the meanings indicated in claim 5,
and
E represents halogen, preferably chlorine,
are reacted with 1,2,3-triazole in inert solvents, if appropriate in the presence of an acid-binding agent,
or
[B] compounds of the general formula (III) 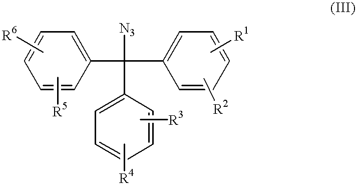
in which
R¹, R², R³, R⁴, R⁵ and R⁶ have the meanings indicated in claim 5, are reacted with acetylene, if appropriate in the presence of a solvent.

8. Compounds according to claim 5 for use as medicaments for the control of disorders of the CNS.

9. Pharmaceutical preparations comprising at least one compound of the general formula (I) according to claim 1, if appropriate one or more inert, non-toxic, pharmaceutically suitable auxiliaries and excipients and, if appropriate, further pharmaceutical active compounds.
